(19) United States
(12) Patent Application Publication
(10) Pub. No.: US 2018/0318537 A1
(43) Pub. Date: Nov. 8, 2018

(54) EMERGENT CRICOTHYROTOMY GUIDE AND CRADLE

(71) Applicant: Trenton Ames Stephenson, Allen, TX (US)

(72) Inventor: Trenton Ames Stephenson, Allen, TX (US)

(21) Appl. No.: 15/588,479

(22) Filed: May 5, 2017

(51) Int. Cl. A61M 16/04

(52) U.S. Cl. CPC A61M 16/0488; A61M 2209/06; A61M 16/0472

(57) ABSTRACT

An emergent cricothyrotomy guide and cradle positioned on and affixed to a human neck to secure an endotracheal tube within the trachea during an emergency cricothyrotomy. The emergent cricothyrotomy guide and cradle comprises a horseshoe cradle, a strap fastener, and a split inner locking mechanism and may be packaged in an emergency medical kit with a blade knife, a bougie, and an endotracheal tube. The strap fastener is operable to be tied around the endotracheal tube received within the horseshoe cradle, securing the endotracheal tube within the trachea relative to the emergent cricothyrotomy guide and cradle until a permanent airway can be established.

### TECHNICAL FIELD

The present disclosure generally relates to an emergent cricothyrotomy kit, and more particularly, to an emergent cricothyrotomy guide and cradle that is operable to be positioned on and affixed to a human neck and secure an endotracheal tube within the trachea.

### BACKGROUND

A cricothyrotomy (also called cric, thyrocricotomy, cricothyroidotomy, inferior laryngotomy, intercricothyrotomy, coniotomy or emergency airway puncture) is an emergency procedure that uses an incision made through the skin and cricothyroid membrane to establish a patent airway during certain life-threatening situations, such as airway obstruction by a foreign body, angioedema, or massive facial trauma. A cricothyrotomy is indicated when there is an inability to establish an oral or nasal airway and the patient cannot be ventilated. Advantages over a tracheostomy include that a cricothyrotomy is easier to perform, does not require manipulation of the cervical spine, and is associated with fewer complications. While cricothyrotomies are potentially lifesaving in difficult airway situations, they are only meant to be used as a definitive airway for a limited amount of time.

There are currently many advanced airway devices used to assist with difficult intubations, but when they are unsuccessful, an emergency emergent cricothyrotomy is necessary. The rate of a failed emergency department airway requiring a surgical airway is less than 1% in the general population, but rises to 1-2.8% for all intubation attempts in patients with trauma. In such a case, a cricothyrotomy may be necessary. General indications for a cricothyrotomy include the inability to intubate and inability to ventilate.

A cricothyrotomy is generally performed by making a vertical incision on the skin of the neck just below the laryngeal prominence (Adam's apple), then making another transverse incision in the cricothyroid membrane which lies deep to this point. A tracheostomy tube, or endotracheal tube (“ETT”), with a 5.5 mm to 7 mm internal diameter, is then inserted through the incisions and into the trachea. After that, a cuff of the ETT is inflated, and hopefully, the ETT is secured in its proper place. The person performing the procedure might utilize a gum elastic bougie, which is a semi rigid device consisting of a straight piece of plastic with a one inch tip at a 30-degree angle. A bougie is used to guide placement of the endotracheal tube into the trachea. Confirmation of placement is assessed by bilateral ausculation of the lungs, observation of the rise and fall of the chest and the absence of borborygmi. If performed in a hospital setting, an end-tidal CO2 detector and chest radiograph can also confirm placement in the trachea.

However; the current method for performing a cricothyrotomy involves securing the ETT with medical tape or gauze bandages. Using this method, the ETT is often not properly secured in place and may slip out of the neck, closing the airway, or may be inserted too far, permanently damaging the trachea. In addition, without a standard method of securing the ETT, there are often inconsistent amounts of material to secure the ETT.

Therefore, an improved device for positioning and securing an ETT in its proper location and an emergency medical kit containing said device is desired.

### BRIEF SUMMARY

Disclosed herein is an emergent cricothyrotomy guide and cradle comprising a horseshoe cradle, a strap fastener, and a split inner locking mechanism. In an embodiment, the horseshoe cradle comprises an opening at a cephalad end and an outwardly extending lip at a caudad end, wherein a channel is defined by the opening and ends to an endotracheal tube ring at the caudad end. The strap fastener is affixed between a base of the cradle and the split inner locking mechanism. The fastener extends in a cephalad direction and has “zip tie” mechanism to secure itself around an ETT. The endotracheal tube ring is sized and shaped to receive an endotracheal tube. The strap fastener is operable to be tied around an endotracheal tube received within the endotracheal tube ring, thereby securing the endotracheal tube relative to the emergent cricothyrotomy guide and cradle with the strap fastener and the inner locking mechanism. In an embodiment, the emergent cricothyrotomy guide and cradle is packaged in an emergency medical kit comprising a blade knife, a bougie, and an endotracheal tube.

### BRIEF DESCRIPTION OF THE DRAWINGS

Embodiments are illustrated by way of example in the accompanying figures, in which like reference numbers indicate similar parts, and in which:

FIG. 1 illustrates a front view of an emergent cricothyrotomy guide and cradle, in accordance with one embodiment of the present disclosure;

FIG. 2 illustrates a side view of the emergent cricothyrotomy guide and cradle of FIG. 1, in accordance with one embodiment of the present disclosure;

FIG. 3 illustrates a rear view of the emergent cricothyrotomy guide and cradle of FIGS. 1 and 2, in accordance with one embodiment of the present disclosure;

FIG. 4 illustrates a perspective view of the emergent cricothyrotomy guide and cradle of FIGS. 1-3, in accordance with one embodiment of the present disclosure;

FIG. 5 illustrates an orthogonal view of a sealed emergent cricothyrotomy kit, in accordance with one embodiment of the present disclosure;

FIG. 6 illustrates an orthogonal view of individual components the unsealed emergent cricothyrotomy kit of FIG. 5, in accordance with one embodiment of the present disclosure;

FIG. 7 illustrates a front view of a positioned and affixed emergent cricothyrotomy guide and cradle receiving an endotracheal tube, in accordance with one embodiment of the present disclosure; and

FIG. 8 illustrates a side view of the positioned and affixed emergent cricothyrotomy guide and cradle receiving the endotracheal tube of FIG. 7, in accordance with one embodiment of the present disclosure.

### DETAILED DESCRIPTION

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not limit the scope of the disclosure.

To facilitate the understanding of this disclosure, a number of anatomic terms are defined below. Terms defined herein have meanings as commonly understood by a person of ordinary skill in the areas relevant to the present disclosure. “Cephalad” means towards the head. “Caudad” means towards the feet. “Distal” means away from the centerline of the body. “Proximal” means towards the centerline of the body. “Left” is from the perspective of the patient and is their left. “Right” is from the perspective of the patient and is their right. Terms such as “a”, “an”, and “the” are not intended to refer to only a singular entity, but include the general class of which a specific example may be used for illustration. The terminology herein is used to describe specific embodiments of the disclosure, but their usage does not limit the disclosure, except as outlined in the claims.

Various embodiments of an emergent cricothyrotomy kit are described. It is to be understood, however, that the following explanation is merely exemplary in describing the devices and methods of the present disclosure. Accordingly, several modifications, changes, and substitutions are contemplated.

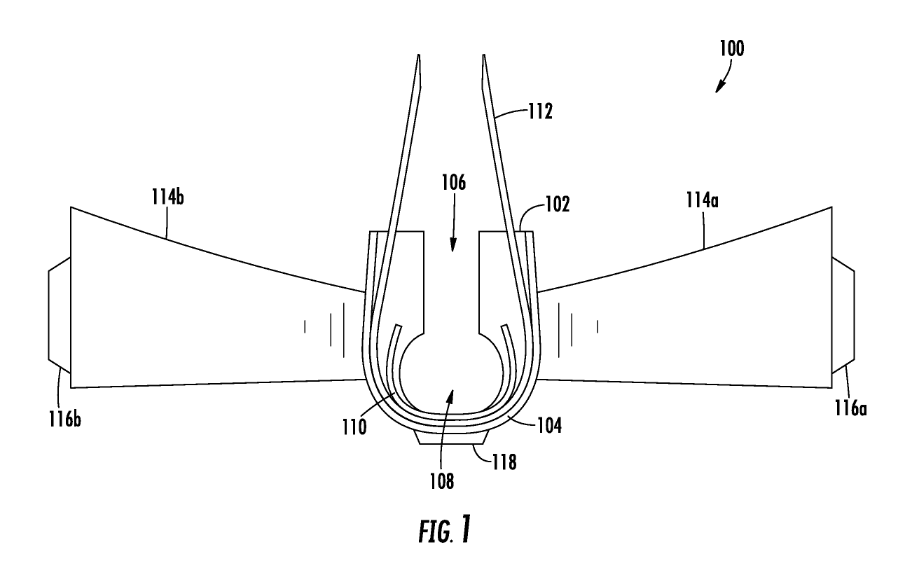

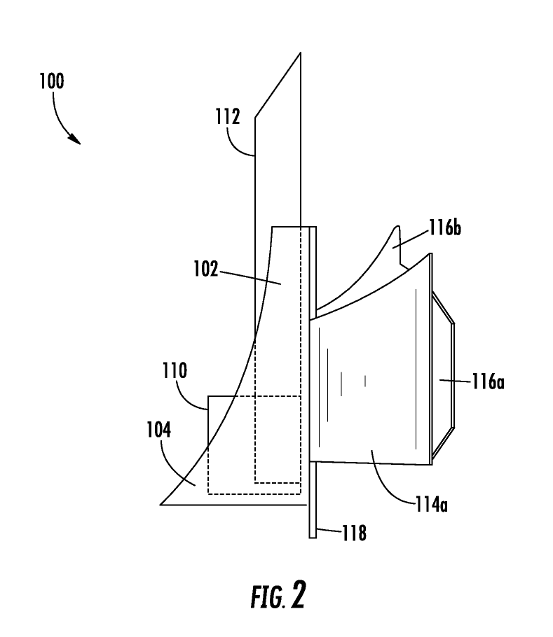

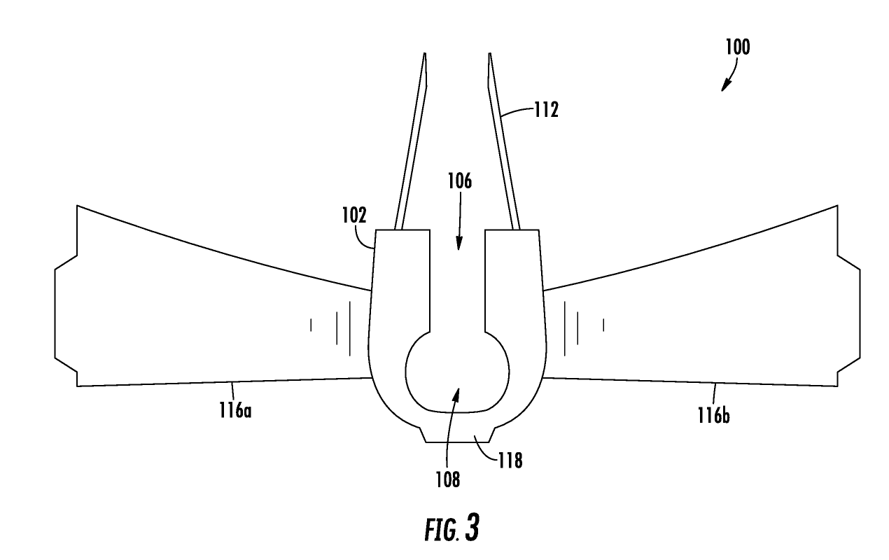

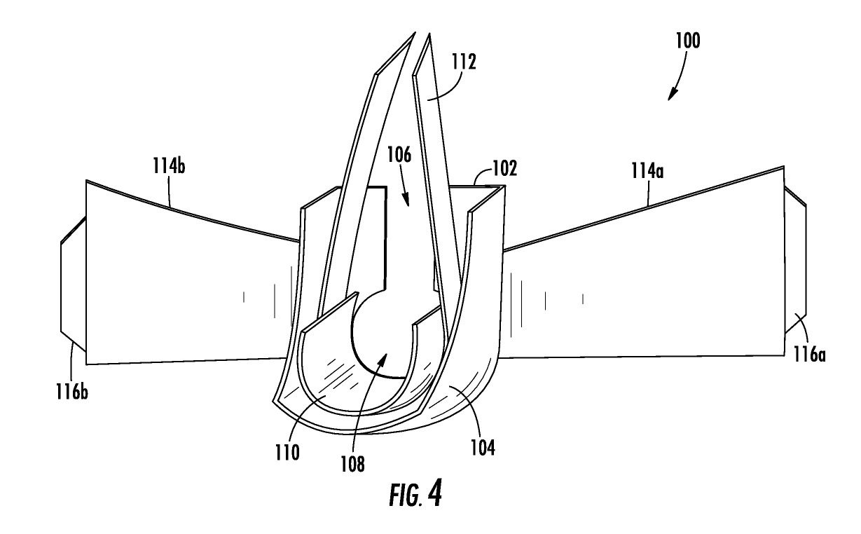

FIG. 1 illustrates a front view of an emergent cricothyrotomy guide and cradle 100, in accordance with one embodiment of the present disclosure. FIG. 2 illustrates a side view of the emergent cricothyrotomy guide and cradle 100 of FIG. 1, in accordance with one embodiment of the present disclosure. FIG. 3 illustrates a rear view of the emergent cricothyrotomy guide and cradle 100 of FIGS. 1 and 2, in accordance with one embodiment of the present disclosure. FIG. 4 illustrates a perspective view of the emergent cricothyrotomy guide and cradle 100 of FIGS. 1-3, in accordance with one embodiment of the present disclosure.

The emergent cricothyrotomy guide and cradle 100 depicted in FIGS. 1-4 comprises a horseshoe-shaped cradle 102 with an outwardly extending lip 104 at a caudad end and an opening 106 at a cephalad end. The opening 106 begins at the cephalad end and extends towards the caudad end, creating a channel for an ETT to be received within the cradle 102. The channel ends at an ETT ring 108 near the caudad end and proximate the outwardly extending lip 104. The channel 106 may be an opening at the cephalad end sized and shaped to allow an ETT to be received within the ETT ring 108. The outwardly extending lip 104 may be used as a lip that an ETT may rest on, thereby reducing tension on the endotracheal tube and the incision in the neck.

In an embodiment, a standard ETT 6.0 tube has a 6.0 mm inner diameter and an 8.8 mm outer diameter. As such, the diameter of the ETT ring 108 may also be 8.8 mm. However, the ETT ring 108 may be sized and shaped to receive any size of known ETT. The width of the channel beginning at the opening 106 may be smaller than the diameter of an ETT and may be sized to receive the caudad end of the laryngeal prominence within the channel, helping to position the emergent cricothyrotomy guide and cradle 100 in the correct location. In an embodiment, the diameter of opening of the ETT ring 108 may be 8.8 mm while the width of the channel may be 8.0 mm or less. By making the width of channel smaller than the diameter of the ETT, once the emergent cricothyrotomy guide and cradle 100 is positioned, the ETT may be secured in place and may not slide in a cephalad direction out the channel opening 106 and relative to the cradle 102.

The cradle 102 may be manufactured from a medical-grade flexible plastic, thereby allowing the cradle 102 to contour to the patient's neck proximate the laryngeal prominence. The flexible plastic also allows the ETT to slide within the channel from the opening 106 to the ETT ring 108 with sufficient force, but prevents the ETT from sliding back towards the opening 106 once it is received within the ETT ring 108. The ETT “snaps” into place within the ETT ring 108, providing a first means of securing the ETT within the ETT ring 108.

The emergent cricothyrotomy guide and cradle 100 may further comprise a tube fastener 112 affixed between a base of the outwardly extending lip 104 and a split inner locking mechanism 110.

The inner locking mechanism 110 may be affixed to an upper surface of the fastener 112. The inner locking mechanism 110 may comprise two wings that create an opening at the cephalad end that is the same size or larger than the channel and ETT ring 108, thereby allowing an ETT to be received through the inner locking mechanism 110 and within the ETT ring 108. The inner locking mechanism may be manufactured from a flexible plastic that may be similar in material consistency to the cradle 102.

The fastener 112 extends in a cephalad direction above the split outwardly extending lip 104 and may be used to secure an ETT. The fastener 112 may comprise or one or more straps and may be manufactured from a medical-grade flexible rubber similar in nature to a goggle strap. The fastener 112 may be sized and shaped to be easily tied with either one or two hands during an emergency procedure. Because of the medical environment, however, it is likely that the fastener 112 will not be manufactured with a natural rubber latex. In another embodiment, the fastener 112 may comprise a “zip tie” mechanism that ratchets around the ETT, securing it in place.

Both the fastener 112 and the inner locking mechanism 110 may be affixed proximate to the base of the ETT ring 108 and above the cephalad surface of the outwardly extending lip 104 such that when an ETT is received within the ETT ring 108, the straps of the fastener 112 may be tied around the ETT, providing a second means of securing the ETT within the ETT ring 108. When the straps of the fastener 112 are tied or knotted around the ETT, the wings of the inner locking mechanism 110 are deformed downwardly around the ETT beneath the straps of the fastener 112, causing the inner locking mechanism 110 to act as a washer of sorts between the fastener 112 and the ETT, providing additional securement of the ETT.

In an embodiment, the emergent cricothyrotomy guide and cradle 100 may further comprise left and right securement wings 114a, 114b, respectively. The securement wings 114a, 114b may be manufactured from a medical-grade flexible plastic, thereby allowing the securement wings 114a, 114b to contour rearwardly to the patient's neck. The securement wings 114a, 114b may be sized and shaped to contour around the patient's neck. In an embodiment, the securement wings 114a, 114b may shaped like a bow-tie, with relatively straight caudad edges, which remain above the left and right clavicles when the emergent cricothyrotomy guide and cradle 100 is positioned on the neck, and curved cephalad edges, which more closely follow the contours of the neck. In an embodiment, the securement wings 114a, 114b may be approximately 10 cm long and 4 cm tall.

In an embodiment, the left and right securement wings 114a, 114b may be sized like “bat wings” to contour around the patient's neck. The securement of the wings 114a, 114b may be received within the anatomical contours of the neck with the cephalad portion being concave in order to fit the convex shape of the neck.

In an embodiment, the left and right securement wings 114a, 114b may further comprise openings (not shown) to allow Velcro straps (not shown) to be connected to the distal ends of the securement wings 114a, 114b. These Velcro straps may loop around the patient's neck and may be used to further secure the emergent cricothyrotomy guide and cradle 100 in place.

The emergent cricothyrotomy guide and cradle 100 may further comprise adhesive backing that may be used to further secure the emergent cricothyrotomy guide and cradle 100 to the patient's neck. The adhesive backing may be a medical grade adhesive that is covered by a removable paper or plastic backing cover.

The cradle 102 may comprise an adhesive backing that is covered by a backing cover 118. The backing cover 118 may be sized and shaped to correspond to the size and shape of the cradle 102 but further comprise one or more tabs extending beyond the cradle 102. The one or more tabs may allow the backing cover 118 to be easily removed from the cradle 102, thereby exposing the adhesive backing and allowing the cradle 102 to be adhesively secured to the neck in the proper position proximate to the Adam's apple.

The left and right securement wings 114a, 114b may comprise left and right adhesive backing that is covered by backing covers 116a, 116b, respectively. The backing covers 116a, 116b may be sized and shaped to correspond to the size and shape of the left and right securement wings 114a, 114b but further comprise one or more tabs extending beyond the left and right securement wings 114a, 114b. These tabs may allow the backing covers 116a, 116b to be easily removed from the left and right securement wings 114a, 114b, thereby exposing the adhesive backing and allowing the left and right securement wings 114a, 114b to be adhesively secured to the neck. In an embodiment, the left and right backing covers 116a, 116b may first be removed in proximal or medial portions and then pulled distally out, similar to removing the backing of a Band-Aid®, thereby exposing and securing the adhesive backing to the patient's neck.

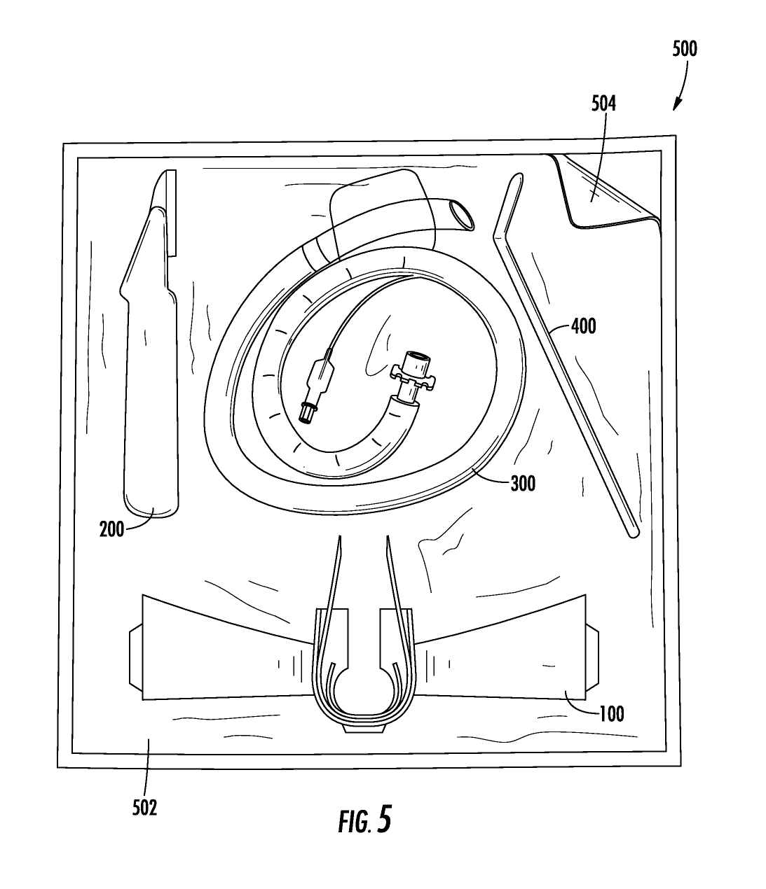

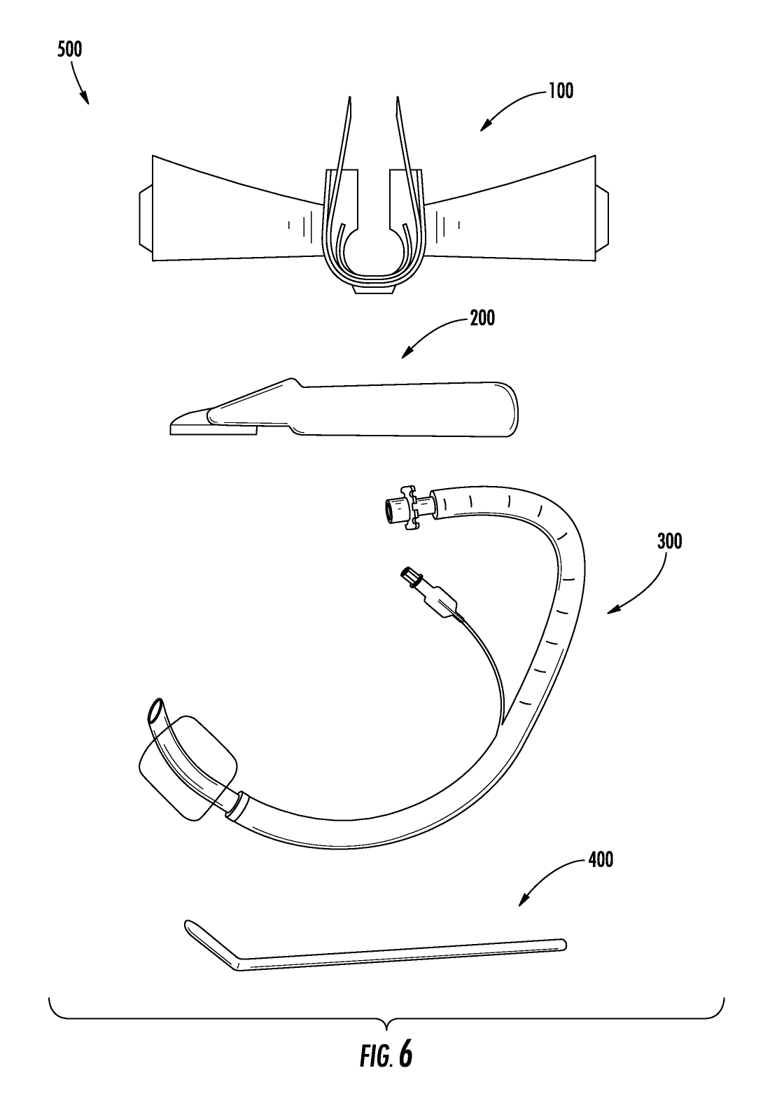

FIG. 5 illustrates an orthogonal view of a sealed emergent cricothyrotomy kit 500, in accordance with one embodiment of the present disclosure. FIG. 6 illustrates an orthogonal view of individual components the unsealed emergent cricothyrotomy kit 500 of FIG. 5, in accordance with one embodiment of the present disclosure.

The kit 500 may comprise a one-time, sterile, sealable bag 502 that may be opened by a tab 504. The tab 504 may be located at one or more corners of the sealable bag 502, allowing the sterile components inside to be accessed when the sealable bag 502 is opened.

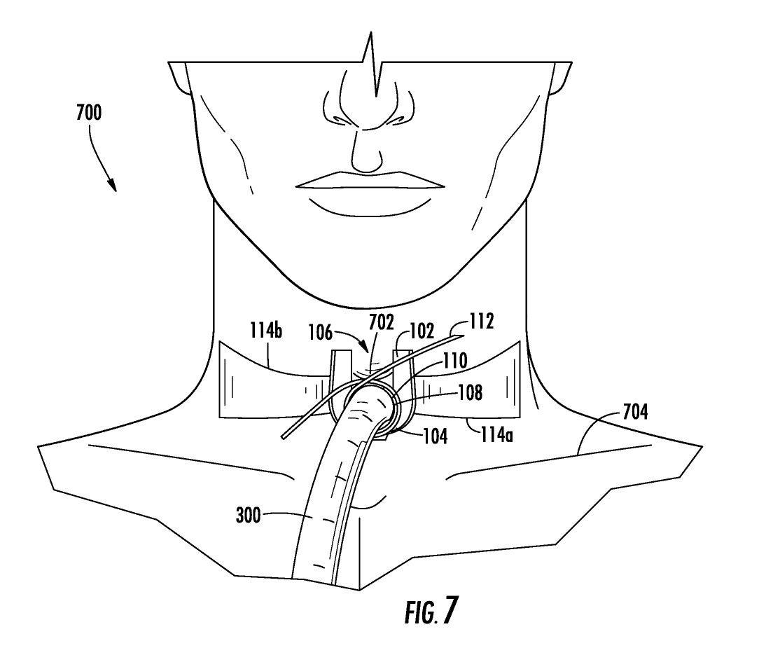

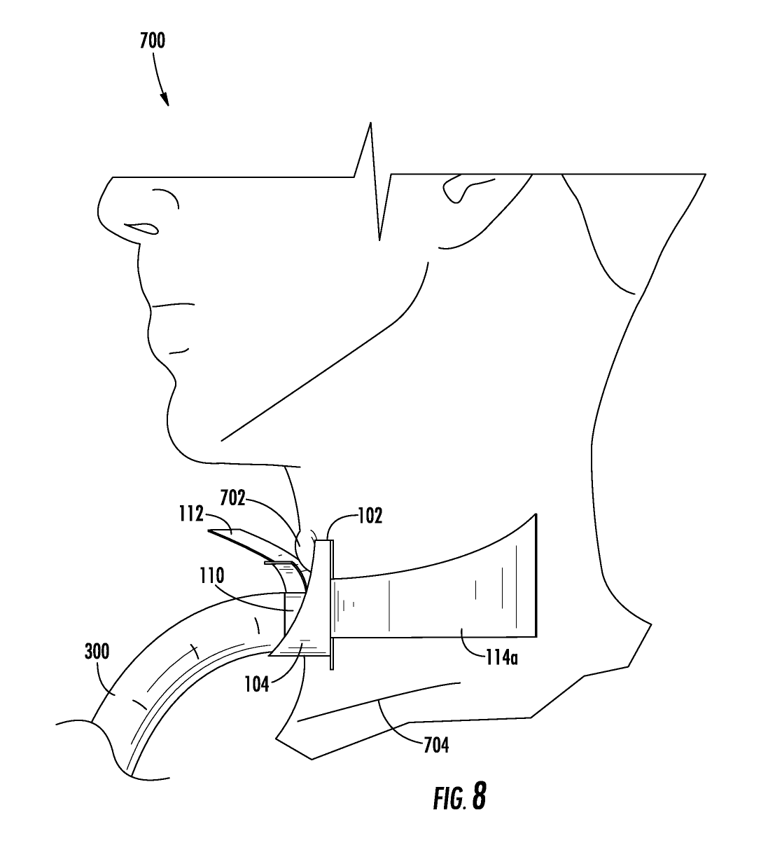

The kit 500 may comprise the emergent cricothyrotomy guide and cradle 100 discussed in FIGS. 1-4, a standard #11 blade knife, a standard 6.0 mm ETT 300, and a standard bougie 400. Each of these components may be used during a life-saving emergent cricothyrotomy procedure, as discussed below in FIGS. 7 and 8. When combined into the sterile kit 500, all components necessary to perform an emergent cricothyrotomy are stored together in an easy-to-find location.

Advantageously, the kits 500 may be purchased and stored anywhere where a first aid kid is located. For example, ideal locations for the kits 500 include ambulances, fire trucks, hospitals, nursing homes, military bases, on military equipment, schools, recreational facilities, sports stadiums, etc. Additionally, the emergent cricothyrotomy guide and cradle 100 may be purchased and stored separately.

FIG. 7 illustrates a front view of a positioned and affixed emergent cricothyrotomy guide and cradle receiving an endotracheal tube 700, in accordance with one embodiment of the present disclosure. FIG. 8 illustrates a side view of the positioned and affixed emergent cricothyrotomy guide and cradle receiving the endotracheal tube 700 of FIG. 7, in accordance with one embodiment of the present disclosure.

In operation, if a patient cannot be intubated by oral or nasal methods and cannot be ventilated, an emergent cricothyrotomy may be necessary to obtain an airway. A cricothyrotomy is a definitive airway and can typically be used up to 24 hours or until a permanent tracheostomy is possible.

If a cricothyrotomy is necessary, a sealed, sterile kit (not shown), may be opened, allowing access to the emergent cricothyrotomy guide and cradle, the standard #11 knife, the standard 6.0 mm ETT, and the standard bougie.

Next, anatomic landmarks may be identified. Below an Adam's apple 702, the thyroid cartilage (the first prominent landmark on the anterior neck), the cricoid cartilage (caudal to the thyroid cartilage), and the area between them, which is the cricothyroid space that contains the membrane need to be palpated. With the nondominant hand, the area to either side of the thyroid cartilage may be stabilized with the first and third fingers, leaving the index finger to palpate the membrane.

Next, a midline vertical incision, approximately 3 cm long and skin deep, may be made with the #11 knife over the cricothyroid membrane with a dominant hand. A midline vertical incision may result in a small amount of venous bleeding but avoids the laterally located vasculature of the neck.

Next, the cricothyroid membrane may be palpated through the incision, using the index of the nondominant hand. A horizontal stab incision may be made through the membrane with the #11 knife. A distinct pop may be felt as the scalpel pierces the membrane and enters the trachea.

Next, the ETT 300 may be inserted into the opening made in the cricothyroid membrane with the dominant hand while directing caudad. The bougie 400 may be used to facilitate the ETT 300 going into the cricothyroid membrane as well. If the bougie 400 is used, it will be placed before the ETT 300, directing in the same caudad fashion. Once the bougie 400 is in place, then the ETT 300 can slide over the bougie 400 in a seldinger fashion, with the ETT 300 going into the trachea as the bougie 400 is removed.

Next, a cuff at the end of the ETT 300 within the trachea may be filled with 5-10 mL of air and the other end of the ETT 300 may be attached to a bag valve mask (“BVM”) and ventilated.

At this step, the ETT 300 is positioned within the trachea, but it is not secure. The ETT 300 needs to be secured before transportation to a hospital or taken to an operating room for a permanent tracheostomy.

Next, the emergent cricothyrotomy guide and cradle 100 is removed from the kit and the adhesive backings (not shown) are removed from the cradle 102 and the left and right securement wings 114a, 114b. Carefully, the opening 106 of the cradle is directed cephalad along the already placed ETT 300 until it snaps into the ETT ring 108. At this point, the emergent cricothyrotomy guide and cradle 100 can be slid down the ETT 300 towards the patient until the emergent cricothyrotomy guide and cradle 100 is in contact with the patient's neck.

Next, the emergent cricothyrotomy guide and cradle 100 may be adhesively secured to the patient's neck with the left and right securement wings 114a, 114b above the clavicles 704 and extending rearwardly around the patient's neck. Once the emergent cricothyrotomy guide and cradle 100 is adhesively secured to the patient's neck, the strap fasteners 112 may be tied around the ETT 300, bending the inner locking mechanism 110 around the ETT 300, thereby creating an additional means of securement. Once secured, the ETT 300 may rest on the outwardly extending lip 104, thereby taking additional pressure off the ETT 300 and the incisions through which it is inserted.

In an embodiment, instead of creating the incisions in the neck and then placing and securing the emergent cricothyrotomy guide and cradle 100, the emergent cricothyrotomy guide and cradle 100 may be positioned and adhered to the neck before the incisions are made. Using this method, the emergent cricothyrotomy guide and cradle 100 may be positioned with the Adam's apple 702 proximate to the opening 106 of the cradle 102. Once adhered to the neck, the ETT ring 108 should provide a guide for where to make the incisions in the neck with the #11 blade knife.

Once the incisions are made, the ETT 300 may be inserted into the trachea in the same method as described above. Additionally, the ETT 300 may be secured with the fasteners 112 in the same method as described above.

While various embodiments in accordance with the principles disclosed herein have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

It will be understood that the principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. An emergent cricothyrotomy guide and cradle comprising:
a horseshoe cradle comprising an opening at a cephalad end and an outwardly extending lip at a caudad end, wherein a channel is defined by the opening and ends at an endotracheal tube ring at the caudad end, and wherein the opening of the horseshoe cradle is sized and shaped to conform around a laryngeal prominence and provide a guide for a cricothyrotomy incision;
a strap fastener affixed to a base of the outwardly extending lip; and
an inner locking mechanism comprising a unitary body and two wings creating an opening at the cephalad end and affixed to an upper surface of the fastener;
wherein the endotracheal tube ring is sized and shaped to receive an endotracheal tube;
wherein the strap fastener is operable to be tied around an endotracheal tube received within the endotracheal tube ring, thereby securing the endotracheal tube relative to the emergent cricothyrotomy guide and cradle with the strap fastener; and
wherein the wings of the inner locking mechanism are operable to be deformably bent around the endotracheal tube, thereby providing additional securement of the endotracheal tube relative to the emergent cricothyrotomy guide and cradle with the inner locking mechanism.

2. The emergent cricothyrotomy guide and cradle of claim 1, wherein the cradle is manufactured from a flexible plastic.

3. The emergent cricothyrotomy guide and cradle of claim 1, wherein the strap fastener is manufactured from a rubber.

4. The emergent cricothyrotomy guide and cradle of claim 1, wherein the inner locking mechanism is manufactured from a flexible plastic.

5. The emergent cricothyrotomy guide and cradle of claim 1, further comprising left and right securement wings affixed to the distal sides of the cradle.

6. The emergent cricothyrotomy guide and cradle of claim 5, wherein the left and right securement wings are manufactured from flexible plastic.

7. The emergent cricothyrotomy guide and cradle of claim 5, wherein the left and right securement wings are bowtie shaped.

8. The emergent cricothyrotomy guide and cradle of claim 5, wherein the left and right securement wings are bat wing shaped with a straight edge at a caudad end and an upwardly curving edge at a cephalad end.

9. The emergent cricothyrotomy guide and cradle of claim 5, wherein the left and right securement wings further comprise apertures at the distal ends of the securement wings operable to receive a hook and loop fastener strap.

10. The emergent cricothyrotomy guide and cradle of claim 1, wherein the cradle further comprises an adhesive backing on a rear surface opposite the outwardly extending lip operable to affix to a patient's neck.

11. The emergent cricothyrotomy guide and cradle of claim 10, wherein the adhesive backing is covered with a removable adhesive backing cover.

12. The emergent cricothyrotomy guide and cradle of claim 5, wherein the left and right securement wings further comprise adhesive backing on rear surfaces operable to affix to a patient's neck.

13. The emergent cricothyrotomy guide and cradle of claim 12, wherein the adhesive backings are covered with removable adhesive backing covers.

14. The emergent cricothyrotomy guide and cradle of claim 1, wherein the endotracheal tube ring is sized and shaped to receive an endotracheal tube with an inner diameter of 6.0 mm.

15. The emergent cricothyrotomy guide and cradle of claim 1, wherein a width of the channel is less than a diameter of the endotracheal tube ring.

16. A emergent cricothyrotomy kit, the kit comprising:
a blade knife;
a bougie;
an endotracheal tube; and
an emergent cricothyrotomy guide and cradle comprising:
a horseshoe cradle comprising an opening at a cephalad end and an outwardly extending lip at a caudad end, wherein a channel is defined by the opening and ends at an endotracheal tube ring at the caudad end, and wherein the opening of the horseshoe cradle is sized and shaped to conform around a laryngeal prominence and provide a guide for a cricothyrotomy incision;
a strap fastener affixed to a base of the outwardly extending lip; and
an inner locking mechanism comprising a unitary body and two wings creating an opening at the cephalad end and affixed to an upper surface of the fastener;
wherein the endotracheal tube ring is sized and shaped to receive an endotracheal tube;
wherein the strap fastener is operable to be tied around the endotracheal tube received within the endotracheal tube ring, thereby securing the endotracheal tube relative to the emergent cricothyrotomy guide and cradle with the strap fastener and the inner locking mechanism; and
wherein the wings of the inner locking mechanism are operable to be deformably bent around the endotracheal tube, thereby providing additional securement of the endotracheal tube relative to the emergent cricothyrotomy guide and cradle with the inner locking mechanism.

17. The emergent cricothyrotomy kit of claim 16, further comprising a one-time, sterile, sealable bag.

18. The emergent cricothyrotomy kit of claim 16, wherein the emergent cricothyrotomy guide and cradle further comprises left and right securement wings affixed to the distal sides of the cradle.

19. The emergent cricothyrotomy kit of claim 18, wherein the cradle and the left and right securement wings further comprise adhesive backings on rear surfaces opposite the outwardly extending lip and operable to affix to a patient's neck.

20. A method for securing an endotracheal tube received within a trachea, the method comprising:
providing an emergent cricothyrotomy guide and cradle, wherein the emergent cricothyrotomy guide and cradle comprises:
a horseshoe cradle comprising an opening at a cephalad end and an outwardly extending lip at a caudad end, wherein a channel is defined by the opening and ends at an endotracheal tube ring at the caudad end, and wherein the opening of the horseshoe cradle is sized and shaped to conform around a laryngeal prominence and provide a guide for a cricothyrotomy incision;
a strap fastener affixed to a base of the outwardly extending lip;
an inner locking mechanism comprising a unitary body and two wings creating an opening at the cephalad end and affixed to an upper surface of the fastener; and
left and right securement wings affixed to the distal sides of the cradle;
wherein the cradle and the left and right securement wings further comprise adhesive backings on rear surfaces opposite the outwardly extending lip operable to affix to a patient's neck;
wherein the adhesive backings are covered with removable adhesive backing covers;
removing the adhesive backing covers, thereby exposing the adhesive backing on the rear surfaces of the cradle and left and right securement wings;
receiving the endotracheal tube within the opening of the cradle and sliding the endotracheal tube down the channel of the cradle to the endotracheal tube ring; and
tying the strap fastener around the endotracheal tube received within the endotracheal tube ring, thereby deformably bending the wings of the inner locking mechanism around the endotracheal tube and securing the endotracheal tube relative to the emergent cricothyrotomy guide and cradle with the strap fastener and the inner locking mechanism.
